(19) United States
(12) Patent Application Publication
(10) Pub. No.: US 2014/0057976 A1
(43) Pub. Date: Feb. 27, 2014

(54) USNIC ACID TOPICAL FORMULATION

(71) Applicants: Steven John Abbott, Yorkshire (GB); Gavin Donoghue, Yorkshire (GB); Elizabeth Anne Eady, Yorkshire (GB)

(72) Inventors: Steven John Abbott, Yorkshire (GB); Gavin Donoghue, Yorkshire (GB); Elizabeth Anne Eady, Yorkshire (GB)

(73) Assignee: EVOCUTIS PLC, Yorkshire (GB)

(21) Appl. No.: 14/008,376

(22) PCT Filed: Mar. 26, 2012
(86) PCT No.: PCT/GB2012/050664

§ 371 Date: Nov. 15, 2013

(30) Foreign Application Priority Data
Mar. 31, 2011 (GB) 1105410.3
Jun. 29, 2011 (GB) 1111013.7

(51) Int. Cl. A61K 31/343

(52) U.S. Cl. CPC A61K 31/343
USPC 514/468

(57) ABSTRACT

Topical skin treatment formulation containing usnic acid or an usnate salt, dissolved in a solvent system comprising (i) dimethyl isosorbide; (ii) a C1 to C9 alkyl salicylate; and (iii) a glyceryl fatty acid ester. The solvent system may also comprise an alcohol, a polyoxyalkylene-based solvent, and/or a C1 to C4 alkyl glucose ester. The formulation may be used in the treatment of microbial conditions, in particular acne. The solvent system assists in the effective dissolution of the usnic acid or usnate and in targeting its delivery to relevant sites on the skin.

### FIELD OF THE INVENTION

This invention relates to topical skin treatment formulations containing usnic acid or usnate salts, and to the preparation and use of such formulations.

### BACKGROUND TO THE INVENTION

Usnic acid and usnate salts are known for use as topical antimicrobial agents. Some, for example copper usnate, have been found to be active against P. acnes, the bacteria implicated in inflammatory acne, and have thus been suggested for use as topical anti-acne agents.

However, usnic acid and usnates can be difficult to formulate in a manner which delivers them efficiently to target sites in the skin. They are typically insoluble in water and in many other commonly used solvents, and can suffer from instability in certain solvent environments: it is not therefore straightforward to identify suitable skin-compatible vehicles in which to deliver the actives. The insolubility of the usnates also limits their ability to penetrate the skin, and thus to reach the pilosebaceous follicles in which the P. acnes bacteria reside.

Previous attempts to target topically delivered active substances to the pilosebaceous follicles have often been directed at transepidermal delivery, and have focused on improving the rate at which the formulated substances can penetrate and diffuse through the various layers of the skin (the stratum corneum, the epidermis and the dermis) and thence into the follicles. To this end, additives such as skin penetration enhancers and diffusion coefficient enhancers have often been added to the formulations, or the solvents used have been selected for their ability to diffuse quickly through the relevant skin layers. Partition coefficient enhancers have also been included, in order to improve partitioning of the active substances between their formulations and the skin. The resultant formulations have typically been relatively complex both to design and to manufacture. Moreover these attempts have met with only limited success and as a result, active substances often need to be formulated at relatively high concentrations to ensure that a sufficient quantity reaches the intended site of action. Again this is an issue for substances, such as usnic acid and usnates, which are poorly soluble in commonly-used skin delivery vehicles and are therefore difficult to formulate at effective doses.

Some of the larger usnates (in particular di-usnates such as copper (II) di-usnate, which has a molecular weight of greater than 700) can be particularly hard to formulate at sufficiently high concentrations.

Thus, many conventional approaches to formulating active substances for topical delivery, and in particular for use as anti-acne agents, are likely to prove ineffective for usnic acid and usnate salts. Indeed this has been found to be the case by the present inventors.

It is an aim of the present invention to provide novel topical skin treatment formulations containing usnic acid or usnate salts, which formulations can overcome or at least mitigate the above described problems. Ideally such formulations will be capable of delivering the usnic acid or usnate in an efficient and targeted manner, in particular to the pilosebaceous follicles.

### STATEMENTS OF THE INVENTION

According to a first aspect of the present invention there is provided a topical skin treatment formulation containing usnic acid or an usnate salt dissolved in a solvent system, wherein the solvent system comprises:
(i) dimethyl isosorbide (DMI);
(ii) a C1 to C9 alkyl salicylate; and
(iii) a glyceryl fatty acid ester.

This specific combination of solvents has surprisingly been found capable of delivering an usnate salt to the pilosebaceous follicles following topical application to the skin surface, and appears capable of overcoming many of the problems previously associated with the formulation of such actives. The invented formulation has been found to be effective as a topical antimicrobial agent, including against P. acnes, and is thus expected to be of use as an anti-acne agent.

Relatively few individual solvents have been found to be suitable for dissolving usnic acid and usnate salts, much less for transporting such actives through the skin to the target site of action. As described above, these actives cannot readily penetrate through the skin unless carried in a suitable solvent system. According to the present invention, it is necessary to use a specific mixture of solvents which together dissolve the usnic acid or usnate, and deliver it effectively through the skin following topical application, and which are also acceptable for pharmaceutical and/or cosmetic use.

It is believed, although we do not wish to be bound by this theory, that a formulation according to the invention can allow the usnic acid or usnate to diffuse through the skin into the infundibula of the pilosebaceous follicles, where bacteria such as P. acnes reside. Thus, the active substance can be targeted to the follicles, and to micro-organisms which are present in the follicles, via the skin surface.

In an embodiment of the invention, the solvent system comprises, in addition to the components (i) to (iii):
(iv) an alcohol.

In an embodiment, the solvent system comprises, in addition to the components (i) to (iii) and optionally (iv):
(v) a polyoxyalkylene-based solvent selected from polyoxyalkylene glyceryl esters, polyalkylene glycols and mixtures thereof.

In an embodiment, the solvent system comprises, in addition to the components (i) to (iii) and optionally (iv) and/or (v):
(vi) 2-pyrrolidone or a C1 to C12 alkyl pyrrolidone.

In an embodiment, the solvent system comprises, in addition to the components (i) to (iii) and optionally (iv), (v) and/or (vi):
(vii) a C1 to C4 alkyl glucose ester.

In an embodiment, the solvent system comprises, in addition to the components (i) to (iii) and optionally (iv), (v), (vi) and/or (vii):
(viii) a C1 to C4 alkyl pyruvate.

In some embodiments of the invention, however, it may be preferred for the solvent system not to comprise the pyrrolidone component (vi) and/or the pyruvate component (viii).

The solvents (i) to (iii), optionally together with (iv), (v), (vi), (vii) and/or (viii), can together help to keep the active substance in a soluble form so that it can continue to diffuse through the skin and towards the follicles over a longer period following topical application of the formulation—for example for 4 hours or more, or for 8 or 12 hours or more. They also appear to lend substantivity to the formulation, as demonstrated in the examples below, causing it to linger on the skin—and thus to continue to exert its antimicrobial and anti-acne effects—after application. This in turn allows the formulation to be applied for example twice or even once daily, yet to provide a sustained beneficial effect for an extended period after each application. In an embodiment, therefore, the formulation of the invention is a sustained release and/or a sustained action formulation.

In an embodiment of the invention, the solvent system consists essentially of components (i) to (iii), or of components (i) to (iii) together with one or more of the optional components (iv) to (viii). In an embodiment, the solvent system comprises, or consists essentially of, components (i) to (v), in the absence of components (vi) to (viii). In an embodiment it comprises, or consists essentially of, components (i) to (iv), in the absence of components (v) to (viii). In an embodiment it comprises, or consists essentially of, components (i) to (iii) together with either or both of components (iv) and (v), and optionally also with component (vii). In an embodiment it comprises, or consists essentially of, components (i) to (iii) together with either or both (suitably both) of (A) component (v) and (B) ethanol and/or tetrahydrofurfuryl alcohol (THFA).

The usnic acid or usnate salt is present in the formulation as an active substance. It is typically present as an antimicrobial agent, in particular as an antibacterial agent, more particularly as an agent against propionibacteria such as P. acnes, staphylococci such as S. aureus, and/or coryneform bacteria. It may be present as an anti-acne agent.

An usnate salt may be a metal salt. It may be an alkali metal salt such as sodium usnate or an alkaline earth metal salt such as calcium di-usnate, in particular the former. It may be a salt of a multivalent, in particular divalent, metal such as copper (II): it may therefore be a di-usnate, for example copper (II) di-usnate.

In a specific embodiment of the invention, the usnate salt is selected from copper (II) di-usnate, sodium usnate and mixtures thereof. In another specific embodiment, the usnate salt is copper (II) di-usnate (referred to hereafter simply as “copper usnate”).

The concentration of the usnic acid or usnate salt in the formulation may be 0.01% w/w or greater, or 0.05 or 0.1% w/w or greater, or 0.25 or 0.5% w/w or greater, for example 0.8% w/w or greater or in cases 0.9 or 1% w/w or greater. Its concentration may be up to 5% w/w, or up to 3 or 2% w/w, or up to 1.5 or 1.2 or 1% w/w. In an embodiment, its concentration is from 0.1 to 2% w/w, or from 0.5 to 2% w/w, or from 0.5 to 1.5% w/w, such as about 1% w/w.

The concentration of the DMI in the formulation may be 10% w/w or greater, or 15 or 20% w/w or greater, or 22 or 25 or 26 or 27 or 28% w/w or greater, or in cases 30 or 35% w/w or greater. Its concentration may be up to 35% w/w, or up to 32 or 30 or 29% w/w. In cases it may be up to 40 or even 45% w/w. Its concentration may for example be from 25 to 35% w/w, or from 20 to 30% w/w, or from 25 to 30% w/w, or from 27 to 30 or 33% w/w, such as about 30% w/w. In another embodiment it may be present at about 40% w/w.

The component (ii) is suitably a liquid. It may be an ester of salicylic acid with a suitable alcohol. It may be a C3 to C9 alkyl salicylate or a C5 to C9 alkyl salicylate. The alkyl group may be straight chain or branched, and/or may incorporate one or more cyclic groups. The component (ii) may for example be selected from amyl salicylate, octyl (ethylhexyl) salicylate, homosalate and mixtures thereof. It may in particular be homosalate, which is the ester formed from salicylic acid and 3,3,5-trimethyl cyclohexanol. The concentration of the component (ii) in the formulation may be 5% w/w or greater, or 6 or 7 or 8 or 9 or 9.5% w/w or greater. Its concentration may be up to 13% w/w, or up to 12 or 11 or 10% w/w. Its concentration may for example be from 5 to 10% w/w, or from 6 to 13% w/w, or from 9 to 11% w/w, such as about 9.6 or 10% w/w. In an embodiment, its concentration may for example be from 5 to 12% w/w, or from 6 to 11% w/w, or from 7 to 10% w/w, such as about 8.5% w/w.

The component (iii) is suitably a liquid. It may be for example a glyceryl mono-, di- or tri-fatty acid ester. In one embodiment it is a monoester; in another it is a diester. It may be an ester of a C12 to C22 fatty acid, or of a C12 to C20 fatty acid, or of a C12 to C18 fatty acid, such as a glyceryl oleate or stearate. It may be an L-pyrrolidone carboxylic acid (PCA) glyceryl ester, for example of a C12 to C22 or C12 to C20 or C12 to C18 (in particular C18) fatty acid, for example PCA glyceryl oleate. It may be glyceryl diisostearate. In an embodiment it may be a diglyceride ester, in particular a diglyceride mono- or diester. In an embodiment it may be a polyglyceride ester, in particular a polyglyceride mono- or diester. In an embodiment, it is selected from PCA glyceryl esters (in particular PCA glyceryl oleate), glyceryl diisostearate, and mixtures thereof.

The concentration of the component (iii) in the formulation may be 2% w/w or greater, or 3 or 3.5 or 4 or 4.5 or 5% w/w or greater. Its concentration may be up to 12% w/w, or up to 10 or 8% w/w, or up to 7 or 6.5 or 6 or 5.5 or 5% w/w. Its concentration may for example be from 3 to 7% w/w, or from 4 to 6% w/w, such as about 4.8 or 5% w/w. Where the formulation comprises a mixture of two or more components (iii), the above concentration ranges suitably apply to the overall mixture. Thus, for example, the formulation may contain from 3 to 7% w/w of a first glyceryl fatty acid ester, such as PCA glyceryl dioleate, and from 3 to 7% w/w of a second glyceryl fatty acid ester, such as glyceryl diisostearate.

In an embodiment, in particular although not exclusively when the component (iii) is glyceryl diisostearate, the concentration of the component (iii) in the formulation may be up to 45% w/w, or up to 40% w/w, or up to 35 or 30 or 25 or 20% w/w. In such a case its concentration may for example be 15% w/w or greater, or 18 or 20% w/w or greater, such as from 15 to 40% w/w or from 20 to 40% w/w, in cases about 18 to 20% w/w or about 38 to 40% w/w. A lower concentration of component (iii) may be appropriate in formulations which also contain an alcohol, and/or which contain one or more of the optional components (v) to (viii). Thus, in certain embodiments it may be preferred for the concentration of glyceryl diisostearate in the formulation to be 8% w/w or less, or 7 or 6 or 5% w/w or less, or in cases to be 4 or 3 or 2% w/w or less. In some embodiments, it may be preferred for the formulation not to contain any glyceryl diisostearate.

Where the solvent system comprises PCA glyceryl oleate, it may be preferred that its concentration in the overall formulation be 8% w/w or less, or 7 or 6 or 5% w/w or less, or in cases 4 or 3 or 2% w/w or less. In some embodiments, it may be preferred for the formulation not to contain any PCA glyceryl oleate. In some embodiments, it may be preferred for the formulation not to contain both glyceryl diisostearate and PCA glyceryl oleate.

The alcohol component (iv), if present, may for example be selected from C1 to C3 alcohols such as ethanol and isopropyl alcohol; phenoxyethanol; 1-methoxy-2-propanol; benzyl alcohol; THFA; Transcutol™ (2-(2-ethoxyethoxy)ethanol); and mixtures thereof, or from C1 to C3 alcohols such as ethanol; THFA; phenoxyethanol; benzyl alcohol; and mixtures thereof. In an embodiment the alcohol is a C1 to C3 alcohol, in particular ethanol. In an embodiment it is THFA. In an embodiment it is selected from ethanol, THFA and in particular mixtures thereof.

The concentration of the component (iv) in the formulation, if present, may be 5 or 8 or 10% w/w or greater, or 15% w/w or greater, or 17 or 18 or 19 or 20% w/w or greater. Its concentration may be up to 35 or 30% w/w, or up to 25 or 23 or 22 or 21 or 20% w/w. Its concentration may for example be from 10 to 20% w/w, or from 15 to 25% w/w, or from 15 or 18 to 22% w/w, such as about 19 or 20% w/w. Where the formulation comprises a mixture of two or more alcohol components (iv), the above concentration ranges suitably apply to the overall mixture. It may for example contain from 5 to 20% w/w of a first alcohol such as THFA, together with from 2 to 15% w/w of a second alcohol such as ethanol. In an embodiment, it contains from 5 to 15% w/w, or from 7 to 13% w/w, of THFA, together with from 5 to 15% w/w, or from 7 to 13% w/w, of ethanol. In some embodiments, it may be preferred for the THFA concentration to be less than 15% w/w, for example 14 or 13 or 12% w/w or less.

In an embodiment, where the solvent system comprises ethanol, the concentration of the ethanol in the overall formulation may be greater than 5% w/w, or greater than 6 or 7 or 8% w/w. However, it may also be preferred to maintain the ethanol concentration at 15% w/w or lower, in particular at 10% w/w or lower, for example at 9 or 8 or 7% w/w or lower.

In an embodiment, where the solvent system comprises THFA, the concentration of the THFA in the overall formulation may be 20% w/w or less, or 17.5 or 15% w/w or less, such as 12.5 or ideally 10% w/w or less.

Where the formulation contains one or more of the optional components (vi) to (viii), in particular two or more of those components, and more particularly all three, it may be preferred for the formulation not to contain the alcohol (iv).

The component (v), if present, is suitably a liquid. In an embodiment, it is a polyoxyalkylene (polyalkoxylated) glyceryl ester, in particular a polyoxyethylene (polyethylene glycol, PEG) glyceryl ester. In an embodiment, it is a polyalkylene glycol, in particular a PEG. It suitably has a molecular weight of 350 or greater, or of 380 or 400 or 450 or greater, for example of 500 or 600 or 700 or 800 or greater.

A polyalkoxylated glyceryl ester (also known as a polyalkoxylated glyceride) is a glyceride ester of a polyalkylene glycol (typically PEG). It may contain mono-, di- or tri-glycerides, partial glycerides or mixtures thereof. It may for example be formed by esterification of a polyalkylene glycol with a glyceride oil of an appropriate chain length. The glyceride components may for example contain from 6 to 20 or from 8 to 18 carbon atoms; they may be selected from caprylic and capric glycerides and mixtures thereof. Suitable such solvents are commercially available as Glycerox™ 767 and Labrasol™, both of which are mixtures of caprylic and capric glycerides of PEGs.

A polyethoxylated glyceryl ester usable as a component (v) in the present invention may be a mixture of PEG-8 caprylic and capric glycerides, such as is commercially available as Labrasol™.

In an embodiment, the component (v) may be or comprise a polyethylene glycol (PEG), suitably a PEG having a molecular weight of 350 or greater, or of 380 or 400 or 450 or greater, for example of from 350 to 450. A suitable example might be a PEG having a molecular weight of about 400.

In an embodiment, the solvent system contains a mixture of two or more components (v). In an embodiment, the component (v) is selected from polyalkoxylated glyceryl esters, polyalkylene glycols (in particular PEGs), and mixtures thereof.

The concentration of the component (v) in the formulation, if present, may be 6% w/w or greater, or 7 or 8 or 9 or 9.5% w/w or greater. Its concentration may be up to 13% w/w, or up to 12 or 11 or 10% w/w. Its concentration may for example be from 6 to 13% w/w, or from 9 to 11% w/w, such as about 9.6 or 10% w/w.

In an embodiment, the component (v) is present in the formulation at up to 40% w/w, or more particularly at up to 35% w/w, or up to 30% w/w, for example from 20 to 30% w/w or from 22 to 32% w/w or from 25 to 30% w/w, such as about 27% w/w. In an embodiment, it is present in the formulation at up to 25% w/w, or up to 20% w/w, such as about 18% w/w. A lower concentration of component (v) may be appropriate in formulations which also contain one or more of the optional components (vi) to (viii).

Where the formulation comprises a mixture of two or more components (v), the above concentration ranges suitably apply to the overall mixture. It may for example contain from 10 to 30% w/w or from 15 to 30% w/w of a first component (v) such as a polyethoxylated glyceryl ester (eg Labrasol™), together with from 1 to 10% w/w of a second component (v) such as a PEG.

The component (vi), if present, is suitably a liquid. It may for example be selected from C1 to C12 alkyl pyrrolidones such as ethyl pyrrolidone; caprylyl pyrrolidone; lauryl pyrrolidone; and mixtures thereof. In an embodiment, it is a C1 to C4 alkyl pyrrolidone, or a C1 to C3 or C1 to C2 alkyl pyrrolidone, in particular ethyl pyrrolidone. In an embodiment, it is 2-pyrrolidone. The concentration of the component (vi) in the formulation, if present, may be 13% w/w or greater, or 15 or 16 or 17 or 18 or 18.5 or 19% w/w or greater. Its concentration may be up to 25% w/w, or up to 24 or 23 or 22 or 21 or 20 or 19.5% w/w. Its concentration may for example be from 15 to 25% w/w, or from 18 to 21% w/w or from 18 to 20% w/w, such as about 19 or 19.1% w/w or about 20% w/w. In an embodiment, it may be preferred for the formulation not to contain the component (vi).

The component (vii), if present, is suitably a liquid. It may be a C1 to C4 alkyl glucose mono-, di- or triester of a C12 to C18 (in particular C18) fatty acid. It may be a C1 to C4 alkyl glucose diester of a C12 to C18 (in particular C18) fatty acid, for example a C1 to C4 alkyl glucose dioleate such as methyl glucose dioleate. It may be a C1 to C2 alkyl glucose ester, for example a methyl glucose ester (which includes a methyl glucose diester). The concentration of the component (vii) in the formulation, if present, may be 2% w/w or greater, or 3 or 3.5 or 4 or 4.5 or 5% w/w or greater. Its concentration may be up to 8% w/w, or up to 7 or 6.5 or 6 or 5.5 or 5% w/w. Its concentration may for example be from 3 to 7% w/w, or from 4 to 5.5% w/w, such as about 4.8 or 5% w/w.

The component (viii), if present, is suitably a liquid. It may be for example methyl pyruvate or ethyl pyruvate, in particular ethyl pyruvate. Its concentration in the formulation may be 5% w/w or greater, or 7 or 10 or 13 or 14% w/w or greater, or in cases 15 or 17 or 18 or 18.5% w/w or greater. Its concentration may be up to 25% w/w, or up to 22 or 20 or 19.5% w/w, or in cases up to 18 or 16 or 15% w/w. Its concentration may for example be from 15 to 25% w/w, or from 18 to 20% w/w, such as about 19 or 19.1% w/w. In an embodiment, its concentration may be from 10 to 20% w/w, or from 12 to 17% w/w, or from 13 to 16 or 14 to 15% w/w, such as about 14.5% w/w.

In an embodiment, a component (viii), in particular ethyl pyruvate, may be combined with an additional solvent such as THFA, for instance in a 50:50 v/v mixture, the concentration of such a mixture being typically as described above for the component (viii) alone. In an embodiment, it may be preferred for the formulation not to contain the component (viii).

The formulation of the invention must be suitable for topical application to the skin, in particular human skin. It may be adapted and/or intended for topical application to the skin. The formulation may be suitable and/or adapted and/or intended for use as a pharmaceutical formulation, or as a cosmetic. In an embodiment it is suitable and/or adapted and/or intended for use as an anti-acne preparation. It may be suitable and/or adapted and/or intended for use in the treatment of one or more of the conditions identified below in connection with the fourth aspect of the invention.

Any component (i) to (viii) included in the formulation should therefore be suitable for topical application to the skin. It should not induce unacceptable levels of irritation on application to the skin, ideally even when the formulation is applied as a “leave-on” treatment. Ideally, such components are acceptable for cosmetic use. Ideally they are acceptable for pharmaceutical (which includes veterinary) use, in particular for pharmaceutical administration to humans.

In an embodiment, the formulation does not contain water, or contains less than 5 or 2 or 1 or 0.5 or 0.1% w/w water. It may for example take the form of a non-aqueous gel. In an embodiment, it does not contain propylene glycol, or contains less than 5 or 2 or 1 or 0.5 or 0.1% w/w propylene glycol. In an embodiment, it does not contain ethanol, or contains less than 10 or 8 or 5 or 2 or 1 or 0.5 or 0.1% w/w ethanol.

The usnic acid or usnate salt should be soluble in the solvent system, by which is meant that the solvent system is able to dissolve the usnic acid or usnate to at least 0.01% w/w, or at least 0.05 or 0.1% w/w, or at least 0.25 or 0.5% w/w, for example to 0.8% w/w or greater or in cases 1 or 1.5 or 2 or 3 or 4% w/w or greater. In an embodiment, the solvent system is able to dissolve the usnic acid or usnate to at least 1% w/w.

In an embodiment, in particular where it is for use in the treatment of acne, the formulation of the invention contains salicylic acid (2-hydroxybenzoic acid) or a derivative thereof. Salicylic acid is a known anti-acne agent which acts as a keratolytic and is widely used to unblock pores to help prevent whiteheads and blackheads becoming inflamed (Waller J M, Dreher F, Behnam S, Ford C, Lee C, Tiet T, Weinstein G D, Maibach H I, “‘Keratolytic’ properties of benzoyl peroxide and retinoic acid resemble salicylic acid in man”, Skin Pharmacol Physiol 2006; 19: 283-9).

A derivative of salicylic acid may in particular be a cosmetically and/or pharmaceutically acceptable derivative. It may be a salt, for example a metal salt or ammonium salt or vitamin salt. Suitable metal salts include the alkali metal salts (for example the sodium and potassium salts, in particular the former) and the alkaline earth metal salts (for example the calcium and magnesium salts, in particular the former). A metal salicylate may also be selected from bismuth salicylate, bismuth subsalicylate and transition metal salts such as zinc, copper or titanium salts. Other salicylate salts include MEA-salicylate and TEA-salicylate.

Other salicylic acid derivatives include salicylic acid esters, in particular alkyl esters (of which the alkyl group may be either straight chain or branched, and/or may incorporate one or more cyclic groups), more particularly C1 to C20 or C1 to C15 or C1 to C10 or C1 to C6, or in cases C12 to C15, alkyl esters, such as in particular methyl salicylate (“wintergreen”), capryloyl salicylic acid and homosalate (3,3,5-trimethylcyclohexyl 2-hydroxybenzoate). Further derivatives include benzyl salicylate and betaine salicylate. In cases they may include substituted salicylic acids and salts and esters thereof, such as 4-amino salicylic acid, thiosalicylic acid and their salts and esters.

A salicylic acid derivative may be selected from 4-aminosalicylic acid and its salts; capryloyl salicylic acid; glucosamine salicylate; MEA-salicylate; TEA-salicylate; metal salicylates such as those listed above; thiosalicylic acid; benzyl salicylate; amyl (pentyl) and isoamyl (isopentyl) salicylates; azeloyl diethyl salicylate; betaine salicylate; butyloctyl salicylate; chitosan salicylate; dipropylene glycol and glycol salicylates; ethylhexyl (octyl) salicylate; hexanediol disalicylate; hexyl dodecylsalicylate; hexyl salicylate; isocetyl salicylate; isodecyl salicylate; menthyl salicylate; methyl salicylate; myristyl salicylate; niacinamide salicylate; phenyl salicylate; potassium methoxysalicylate; pyridoxine salicylate; silanediol salicylate; tridecyl salicylate; trimethylsilyl trimethylsiloxy salicylate; zinc thiosalicylate; 4-acetaminophenyl salicylate; cyclohexanol, 3,3,5-trimethyl-salicylate; sodium ethylmercurithiosalicylate; C12 to C15 alkyl salicylates; isopropylbenzyl salicylate; zinc glycinate salicylate; and mixtures thereof.

In an embodiment of the invention, a salicylic acid derivative may be selected from metal salicylates; alkyl salicylates of chain length C10 or greater; betaine salicylates; TEA-salicylate; MEA-salicylate; and mixtures thereof. In an embodiment, the invented formulation may contain salicylic acid, a metal salicylate, or a mixture thereof. In an embodiment, it contains salicylic acid.

The concentration of the salicylic acid or derivative in the formulation, if present, may be 0.01% w/w or greater, or 0.05 or 0.1% w/w or greater, or 0.25 or 0.5% w/w or greater, for example 0.8% w/w or greater or in cases 0.9% w/w or greater. Its concentration may be up to 10% w/w, or up to 5 or 3% w/w, or up to 2% w/w, or up to 1.5 or 1.2 or 1% w/w. In an embodiment, its concentration is from 0.5 to 3% w/w, or from 1 to 2% w/w, such as about 1% w/w.

In an embodiment, the formulation of the invention contains a C12 to C20 fatty acid or mixture thereof. It may contain a C12 to C18 fatty acid or mixture thereof, or a C14 to C18 fatty acid or mixture thereof, for example selected from caprylic/capric, lauric, palmitic, stearic, sapienic, arachidic, oleic and linoleic acids and mixtures thereof. One or more of the fatty acids may be a constituent of sebum, in particular human sebum: such acids include palmitic acid, sapienic acid and oleic acid, and others described by Nicolaides N in “Skin Lipids—Their Biochemical Uniqueness”, Science, 1974, 186: 19-26. In an embodiment, the formulation contains oleic acid. The concentration of such a component in the formulation may be 0.05% w/w or greater, for example 0.1 or 0.2 or 0.3 or 0.4 or 0.5% w/w or greater. Its concentration may be up to 5% w/w, or up to 2 or 1% w/w, for example up to 0.9 or 0.8 or 0.7 or 0.6% w/w. Its concentration may be for example from 0.3 to 0.7% w/w, such as about 0.5 or 0.57% w/w. In certain embodiments, however, it may be preferred for the formulation not to contain a C12 to C20 fatty acid.

In an embodiment, the formulation contains L-pyrrolidone carboxylic acid (PCA). The concentration of the PCA in the formulation may be 0.05% w/w or greater, for example 0.1 or 0.2 or 0.3 or 0.4 or 0.5% w/w or greater. Its concentration may be up to 2 or 1.5 or 1% w/w, for example up to 0.9 or 0.8 or 0.7 or 0.6% w/w. Its concentration may be for example from 0.2 to 1.3% w/w, or from 0.3 to 0.7% w/w, or from 0.7 to 1.3% w/w, such as about 0.5% w/w or about 1% w/w. In certain embodiments, however, it may be preferred for the formulation not to contain PCA. In an embodiment, the pH of the formulation—when measured in the presence of water—is from about 3.5 to 5.5, or from about 4 to 5.5. In an embodiment, the pH of the formulation, when measured in the presence of water, is from about 3 to 5 or from about 3 to 4, for example about 3.5.

A formulation according to the invention may also contain an antioxidant. This can help to stabilise the usnic acid or usnate salt, which may be unstable in light and/or susceptible to oxidation in some solvent environments. Oxidative stability can be particularly important for formulations which are intended for use as “leave-on” products, since they can remain on the skin, exposed to both oxygen and sunlight, for extended periods. Antioxidants suitable for use in topical skin treatment formulations are well known to those skilled in the art.

Where the formulation contains an antioxidant, its concentration may be 0.1% w/w or greater, or 0.2 or 0.3 or 0.4 or 0.5% w/w or greater, or in cases 0.6 or 0.7 or 0.8% w/w or greater. Its concentration may be up to 2 or 1.5% w/w, or up to 1.4 or 1.3 or 1.2% w/w, such as about 1% w/w. The formulation may contain one or more additional ingredients or excipients, as are known for use in topical skin treatment formulations. Those included will depend on the intended mode of application for the formulation. Examples may for instance be found in Williams' Transdermal and Topical Drug Delivery (Pharmaceutical Press, 2003) and other similar reference books. See also the Database of Cosmetic and Toiletry Formulations (CD ROM, 2005) by Ernest W Flick, published by William Andrew; the Personal Care Product Council's online database (www.ctfa-online.org); and approved ingredients lists published by regulatory authorities, for example the EU Cosmetic Ingredients list which is available online through the European Commission (see http://ec.europa.eu/enterprise/cosmetics/cosing).

Suitable additives may for instance include emollients, moisturisers, preservatives, stabilisers, gelling agents and other thickeners, sunscreens, colouring agents and fragrances. For use in the treatment of acne, however, it may be preferred for the formulation not to contain an emollient. In a specific embodiment, the formulation contains a fragrance, for example an essential oil or component thereof such as vanillin. In general terms, it may include a component capable of masking, at least partially, the smell of another component present in the formulation.

In an embodiment, the formulation contains one or more thickening agents, which may be gelling agents. Suitable such agents include cellulose-based thickening agents such as methyl cellulose, ethyl cellulose, hydroxyethyl cellulose, hydroxypropyl cellulose, hydroxypropyl methyl cellulose and carboxymethyl celluloses. Such agents may be used in the form of a (preferably cosmetically and/or pharmaceutically acceptable) salt such as for instance the sodium salt. A thickening agent may be a polymeric thickening agent such as a carbomer, which will typically be a cross-linked acrylic acid-based polymer, for example a homopolymer of acrylic acid cross-linked with an allyl ether: such thickeners are available for instance in the Carbopol™ range, ex-Lubrizol.

In an embodiment, the formulation contains a hydroxypropyl cellulose thickening agent, for example of the type commercially available as Klucel™ from Hercules, USA. Such a thickening agent may for example have a molecular weight of 200,000 or greater, or of 300,000 or 350,000 or greater, for example from 300,000 to 500,000 or from 300,000 to 400,000.

Where the formulation contains a thickening agent, its concentration may be 0.2% w/w or greater, or 0.5% w/w or greater, or 1 or 1.5% w/w or greater, or in cases 5% w/w or greater. Its concentration may be up to 10% w/w, or more usually up to 5% w/w, or up to 4 or 3 or 2.5 or 2% w/w, for example from 1 to 2.5% w/w or from 1.5 to 2.5% w/w or from 1.5 to 3% w/w. In an embodiment, the concentration of the thickening agent in the formulation is about 1.9 or 2% w/w, or about 2.5% w/w. In cases it may be used at 1% w/w or less. However, a suitable concentration for a thickening agent (or mixture thereof) will depend on the desired viscosity of the ultimate formulation, and on the properties of the thickening agent as well as any restrictions on its permitted levels in for example cosmetic or pharmaceutical preparations.

The formulation may contain one or more additional active agents such as antimicrobial (in particular antibacterial) agents. For example, it may contain one or more agents selected from anti-acne agents, keratolytics, comedolytics, agents capable of normalising keratinocyte and/or sebocyte function, anti-inflammatories, anti-proliferatives, antibiotics, anti-androgens, sebostatic/sebosuppressive agents, anti-pruritics, immunomodulators, agents which promote wound healing, additional antimicrobial agents, and mixtures thereof.

An additional antimicrobial agent may be selected from biocides, disinfectants, antiseptics, antibiotics, bacteriophages, enzymes, anti-adhesins, immunoglobulins, antimicrobially active antioxidants, and mixtures thereof; it may be active as a bactericide, in particular against propionibacteria and/or staphylococci and/or coryneform bacteria. It may however be preferred for the usnic acid or usnate (and where appropriate the salicylic acid or derivative) to be the only active agent(s) in the formulation, or at least to be the only antimicrobially or antibacterially active agent(s) and/or the only anti-acne active agent(s).

In a specific embodiment, a formulation according to the invention contains usnic acid or an usnate salt dissolved in a solvent system, wherein the solvent system comprises:
(1) DMI, suitably at a concentration from 25 to 35% w/w or from 25 to 30% w/w;
(2) a C5 to C9 alkyl salicylate, in particular homosalate, suitably at a concentration from 5 to 12% w/w;
(3) a glyceryl fatty acid ester selected from PCA glyceryl oleate, glyceryl diisostearate and mixtures thereof, suitably at a concentration from 2 to 8% w/w;
(4) an alcohol selected from ethanol, THFA and mixtures thereof, suitably at a concentration from 15 to 25% w/w;
(5) a component (v) selected from polyoxyethylene glyceryl esters (in particular Labrasol™), PEGs, and mixtures thereof, suitably at a concentration from 22 to 32% w/w; and
(6) a methyl glucose ester, in particular methyl glucose dioleate, suitably at a concentration from 2 to 8% w/w.

Such a formulation suitably also comprises a cellulosic thickening agent, in particular hydroxypropyl cellulose, suitably at a concentration from 1 to 4% w/w. The concentration of the usnic acid or usnate salt in such a formulation is suitably from 0.5 to 1.5% w/w.

Such a formulation also suitably comprises salicylic acid or a salicylate (in particular salicylic acid), suitably at a concentration from 0.5 to 1.5% w/w. It suitably has the form of a gel.

The formulation of the invention may be in the form of a fluid, for example a lotion, cream, ointment, varnish, paste, gel or other viscous or semi-viscous fluid, or a less viscous fluid such as might be used in sprays, foams, drops and dropping fluids, or aerosols. A liquid formulation may itself be formulated as suspended (for example aerosolised) liquid droplets within another fluid carrier.

The formulation may in particular be in the form of a solution, lotion, cream or gel. In an embodiment, it is a cream or gel, in particular a gel. It may comprise a formulation which is, or may be, applied to a carrier such as a sponge, swab, brush, pad, tissue, cloth, wipe, skin patch, dressing (or other material designed for application to the skin), to facilitate its topical administration.

Where the formulation is intended for use in the treatment of body odour, it may contain an anti-perspirant such as an aluminium or aluminium-zirconium salt. It may be in the form of an aerosol, or of a roll-on or “stick” anti-perspirant or deodorant of known type, or of a gel or cream or ointment. Such formulations may contain appropriate conventional liquid or solid carriers and excipients. They may contain anti-perspirant and/or deodorant agents, and/or one or more fragrances.

A formulation according to the invention may be incorporated into, and hence applied in the form of, another product such as a hair care or in particular a skin care preparation (for example a skin cleanser, toner or moisturiser, or a shampoo); a deodorant or anti-perspirant; a cosmetic (eg a makeup product); a cleansing preparation (for example a hand wash or facial wash); a pharmaceutical preparation; a cosmeceutical preparation; or a toiletry product (for instance a bath or shower additive or a soap). The formulation may for example be, or be incorporated into, a “leave-on” skin treatment product.

The invention thus provides, according to a second aspect, a product which incorporates a topical skin treatment formulation according to the first aspect.

A third aspect of the invention provides a method for preparing a topical skin treatment formulation according to the first aspect, the method involving dissolving usnic acid or an usnate salt in a solvent system of the type defined above with reference to the first aspect of the invention. The components of the formulation may be mixed together in conventional manner. For example, the usnic acid or usnate may firstly be dissolved in one or more of the components in which it is relatively freely soluble, prior to mixing with the components in which it is less freely soluble and any other remaining ingredients of the formulation (for example thickeners, fragrances and/or antioxidants). Stirring and/or heating may be used to aid efficient mixing of ingredients, and/or dissolution of the usnic acid or usnate active, at appropriate stages during such a process.

According to a fourth aspect of the invention, there is provided a formulation according to the first aspect, for use in a method of therapy carried out on a living human or animal, in particular human, body. In this context the solvent system should be pharmaceutically acceptable (which includes acceptable for veterinary use).

In an embodiment, the formulation is for use in the treatment of a condition which is caused by, transmitted by and/or exacerbated by (in particular either caused or transmitted by, more particularly caused by) microbial activity. The microbial activity may be bacterial or fungal activity, in particular bacterial activity, more particularly propionibacterial activity.

Such a condition may affect, or be associated with, follicles such as pilosebaceous follicles. For example, the formulation may be for use against a micro-organism which is present in the follicles, such as a staphylococcus or in particular a propionibacterium such as P. acnes, P. granulosum or P. avidum. The condition may affect, or be associated with, the stratum corneum: the formulation may therefore be for use against micro-organisms which are present in the stratum corneum, for example staphylococci or coryneform bacteria.

In an embodiment of the fourth aspect of the invention, the formulation is for use in the treatment of a skin or skin structure condition. Such a condition may be a primary or secondary infection. It may for example be a superficial or uncomplicated skin infection amenable to local therapy. It may be caused, transmitted and/or exacerbated by a Gram-positive bacterium such as a staphylococcus or propionibacterium. It may be acne or an infection associated with acne. It may be a primary or secondary infection due to S. aureus (including MRSA) or a group A beta haemolytic streptococcus. Other skin and skin structure conditions which might be treated according to the invention include infected atopic eczema, superficial infected traumatic lesions, wounds, burns, ulcers, impetigo and folliculitis.

In an embodiment, the formulation is for use in the treatment of acne. Acne is a multifactorial disease of the pilosebaceous follicles of the face and upper trunk, characterised by a variety of inflamed and non-inflamed lesions such as papules, pustules, nodules and open and closed comedones. Its treatment can therefore encompass the treatment (which embraces prevention or reduction) of any of these symptoms, and references to use as an anti-acne agent may be construed accordingly.

In particular, the treatment of acne encompasses the treatment (including prevention) of lesions and/or scarring associated with acne. It also encompasses the inhibition of propionibacterial activity which could cause or be otherwise associated with acne or its symptoms. In the context of the present invention, it may in particular be or involve the treatment of inflamed acne lesions.

Instead or in addition, the formulation of the invention may be for use against an opportunistic infection which is caused, transmitted and/or exacerbated by (in particular caused by) propionibacteria, for instance an infected wound, burn or ulcer. It may be for use against any other infection or condition which involves or can involve propionibacteria, for example body odour.

The formulation may be for use in the treatment (which includes prevention) of a staphylococcal infection, for example due to S. aureus.

The formulation may be for use in the treatment (which includes prevention) of body odour, for example in the axillae or feet. Human body odour is formed by the action of commensal skin bacteria, for example members of the genus Corynebacterium, on the odourless secretions of sweat glands. Other members of the bacterial human skin microflora, such as cutaneous propionibacteria and coagulase negative staphylococci, may also contribute to human body odour. Thus the formulation of the invention may be used against one or more such bacteria.

In the context of the present invention, treatment of a condition encompasses both therapeutic and prophylactic treatment, of either an infectious or a non-infectious condition. It may involve complete or partial eradication of the condition, removal or amelioration of associated symptoms, arresting subsequent development of the condition, and/or prevention of, or reduction of risk of, subsequent occurrence of the condition. It will typically involve use of the formulation as an antimicrobial (in particular antibacterial) and/or anti-acne agent.

Treatment may involve use of the formulation to treat a condition which is caused, transmitted and/or exacerbated by (in particular caused or transmitted by, more particularly caused by), or which is associated with, microbial (in particular bacterial) bio film formation.

The treatment will be administered topically. It may for example involve applying the formulation to the skin daily, or in particular twice daily, as a leave-on formulation. For the treatment of acne, the formulation may be applied to the entire face, or to one or more regions thereof.

A fifth aspect of the invention provides the use of a formulation according to the first aspect, in the manufacture of a medicament (typically a formulation) for the treatment of a skin or skin structure condition, or of a condition which is caused by, transmitted by and/or exacerbated by (in particular either caused or transmitted by, more particularly caused by) microbial activity. The condition may be selected from those listed above in connection with the first to the fourth aspects of the invention. It may in particular be acne, a primary or secondary skin infection, a primary or secondary staphylococcal infection, or body odour. In an embodiment, it is acne.

A sixth aspect provides a method of treatment of a human or animal patient suffering from or at risk of suffering from a skin or skin structure condition, or from a condition which is caused by, transmitted by and/or exacerbated by (in particular either caused or transmitted by, more particularly caused by) microbial activity, the method involving administering to the patient a therapeutically (which term includes prophylactically) effective amount of a formulation according to the first aspect. The formulation may be administered in an antimicrobially effective amount. It should be administered topically.

In an embodiment of the sixth aspect of the invention, the patient is suffering from the relevant condition. In an embodiment, the patient is a human patient. Again, the condition may be selected from those referred to above in connection with the first to the fourth aspects of the invention.

A seventh aspect provides the use of a formulation according to the first aspect of the invention, for non-therapeutic purposes. In an embodiment of this seventh aspect, the formulation is used as an anti-acne or in particular as a skin care agent for non-therapeutic purposes, for example for cosmetic purposes such as to improve the appearance, feel or smell of the skin. It may be used as a toiletry or makeup product.

According to an eighth aspect of the invention, there is provided the use of a formulation according to the first aspect, and/or of a solvent system as defined in connection with the first aspect, for the purpose of improving the delivery of usnic acid or an usnate salt (in particular copper usnate) to a target site in or on the skin. In this context, improving the delivery of the usnic acid or usnate may involve increasing the rate of its delivery; increasing the amount or proportion of it which reaches the target site, or the amount or proportion which reaches the target site within a specified time period; increasing control over the rate or time or quantity of delivery; and/or targeting the delivery more accurately to the target site or to a desired delivery time. Improving the delivery of the usnic acid or usnate may involve enhancing the efficacy or the perceived efficacy of the usnic acid or usnate at the target site, which may involve increasing the speed and/or magnitude and/or duration and/or locus of the effect (typically a therapeutic effect, for example an antimicrobial and/or anti-acne effect) that the usnic acid or usnate has at the target site, or increasing control over the speed, magnitude, timing, duration or locus of the effect. The invention may be used to achieve any degree of improvement in the delivery of the usnic acid or usnate. The target site may in particular be a follicle, more particularly a pilosebaceous follicle. It may be the stratum corneum.

A formulation according to the invention may be marketed with an indication that it provides improved delivery of the usnic acid or usnate contained within it. Such marketing may include an activity selected from (a) enclosing the formulation in a container or package that comprises the relevant indication; (b) packaging the formulation with a package insert that comprises the indication; (c) providing the indication in a publication or sign that describes the formulation; and (d) providing the indication in a commercial which is aired for instance on the radio, television or internet. The improved delivery may be attributed, in such an indication, at least partly to the nature of the solvent system in which the usnic acid or usnate is dissolved. The invention may involve assessing the delivery of the usnic acid or usnate from the formulation during or after its preparation. It may involve assessing the usnic acid or usnate delivery both in the presence and the absence of the solvent system required by the present invention, for example so as to confirm that the solvent system contributes to improved usnic acid or usnate delivery.

According to a further aspect of the invention, there is provided a topical skin treatment formulation containing salicylic acid or a derivative thereof dissolved in a solvent system as defined above in connection with the first aspect of the invention. Such a formulation may, but need not, contain usnic acid or an usnate salt. It is believed that the solvent system is also capable of improving delivery of the salicylic acid or derivative to the skin, and to target sites within the skin such as the pilosebaceous follicles.

HI Throughout the description and claims of this specification, the words “comprise” and “contain” and variations of the words, for example “comprising” and “comprises”, mean “including but not limited to”, and do not exclude other moieties, additives, components, integers or steps. Moreover the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Other features of the invention will become apparent from the following examples. Generally speaking the invention extends to any novel one, or any novel combination, of the features disclosed in this specification (including any accompanying claims and drawings). Thus features, integers, characteristics, compounds, chemical moieties or groups described in conjunction with a particular aspect, embodiment or example of the invention are to be understood to be applicable to any other aspect, embodiment or example described herein unless incompatible therewith. Moreover unless stated otherwise, any feature disclosed herein may be replaced by an alternative feature serving the same or a similar purpose.

Where upper and lower limits are quoted for a property, for example for the concentration of a component in a formulation, then a range of values defined by a combination of any of the upper limits with any of the lower limits may also be implied.

In this specification, references to properties such as solubilities, liquid phases and the like are—unless stated otherwise—to properties measured under ambient conditions, ie at atmospheric pressure and at a temperature of from 18 to 25° C., for example about 20° C.

The present invention will now be further described with reference to the following non-limiting examples.

### DETAILED DESCRIPTION

These experiments involved preparing topical skin treatment formulations according to the present invention, and testing their activity in vivo.

### Example 1

### Formulation A

A gel formulation A according to the present invention was prepared using the components and concentrations listed in Table 1. The first seven components together represented the solvent system in which the copper usnate active was dissolved.

TABLE 1
Formulation A

| | Concentration | |
|---|---|---|
| Component | (% w/w) | Source |
| Dimethyl isosorbide | 28.67 | Sigma-Aldrich, UK |
| (DMI) | | |
| Ethyl pyrrolidone | 19.08 | Sigma-Aldrich, UK |
| Ethyl pyruvate | 19.08 | Sigma-Aldrich, UK |
| Homosalate | 9.6 | TCI Europe |
| Methyl glucose | 4.8 | Surfachem Group Ltd |
| dioleate | | |
| PCA glyceryl oleate | 4.8 | Dr Straetmans Chemische |
| | | Produkte GmbH |
| Labrasol ™ | 9.6 | Gatefosse |
| Copper usnate | 0.99 | Variati |
| PCA | 0.49 | Aston Chemicals |
| Antioxidant | 1 | Sigma-Aldrich, UK |
| Thickener | 1.89 | Honeywill & Stein Ltd |
| TOTAL | 100.0 | |

PCA = L-pyrrolidone carboxylic acid

The formulation was prepared as follows. The copper usnate was premixed with the DMI, the ethyl pyrrolidone, the ethyl pyruvate and the homosalate. The mixture was heated to 40° C. and sonicated for 20 minutes, then filtered to remove any undissolved residue. In a separate container, the Labrasol™, PCA glyceryl oleate and methyl glucose dioleate were mixed together vigorously. This second mixture was then mixed vigorously with the copper usnate-containing premix. To this was added the PCA and the antioxidant, followed by the thickener. The resultant mixture was sonicated at 40° C. for 10 minutes, shaken vigorously and then left to gel for 2 hours at room temperature.

The pH of this formulation, when added to water, was between 3.3 and 3.5.

### Example 2

### Formulation B

An alternative gel formulation B according to the invention was prepared using the components and concentrations listed in Table 2.

TABLE 2
Formulation B

| | Concentration |
|---|---|
| Component | (% w/w) |
| DMI | 28.7 |
| Ethyl pyrrolidone | 19.1 |
| Ethyl pyruvate | 19.1 |
| Homosalate | 9.6 |
| Methyl glucose dioleate | 4.8 |
| PCA glyceryl oleate | 4.8 |
| Labrasol ™ | 9.6 |
| Copper usnate | 0.96 |
| Oleic acid | 0.49 |
| Antioxidant | 0.95 |
| Thickener | 1.90 |
| TOTAL | 100.0 |

The oleic acid was sourced from Sigma-Aldrich, UK.

The formulation was prepared as in Example 1, adding the oleic acid instead of the PCA. Its pH, when added to water, was between 3.3 and 3.5.

### Example 3

### Antimicrobial Activity In Vivo (Formulation A)

This experiment used formulation A as described in Example 1, containing copper usnate as an antimicrobial and anti-acne active substance. Copper usnate is known to be active against the propionibacterium P. acnes NCTC 737, against which it has been found by the present inventors to have a minimum inhibitory concentration (MIC) of 0.49 μg/ml and a minimum biocidal concentration (MBC) of 3.9 μg/ml. It is thus (since the propionibacteria are implicated in acne) known to be of use as an anti-acne agent. It is also known to be active against other bacteria, including staphylococci such as S. aureus.

The formulation was topically applied, twice daily, to three locations on the face of a healthy human volunteer. The skin was sampled before the treatment began, and at 6, 8, 11, 21 and 25 days afterwards whilst the treatment continued, using the method of Williamson-Kligman (Williamson P and Kligman A M, “A New Method for the Quantitative Investigation of Cutaneous Bacteria”, J Invest Dermatol 1965 December; 45(6): 498-503). Samples were collected in the mornings, in each case between 10 and 12 hours after the previously applied evening dose and before application of the morning dose. Samples were also collected two weeks after the last topical application of the formulation.

The samples were assessed for numbers of coagulase-negative staphylococci and Propionibacterium spp by serial dilution and viable counting on suitable media (for the staphylococci, Columbia blood agar (Oxoid) containing 5% defibrinated horse blood (E & O Laboratories), and for the propionibacteria, tryptone (Oxoid, 2%), yeast extract (Oxoid, 1%), glucose (Sigma, 0.5%) agar containing 2 mg/L of furazolidone (Sigma). Colony counts, obtained following incubation at 37° C. aerobically for 24 hours in the case of staphylococci and anaerobically for 7 days in the case of propionibacteria, were used to calculate the log number of bacteria per cm² of skin.

A decline in the numbers of bacteria present at the skin surface, over the course of the treatment period, would indicate that bacteria were being successfully inactivated not only at the skin surface but also in the follicles.

The results are shown in Tables 3a and 3b below, for coagulase-negative staphylococci and propionibacteria respectively.

TABLE 3a
counts of coagulase-negative staphylococci

| Days of | Mean log | Change from |
|---|---|---|
| application | count/cm² skin | baseline |
| 0 (baseline) | 3.82 | n/a |
| 6 | 2.66 | −1.16 |
| 8 | 2.63 | −1.19 |
| 11 | 1.48 | −2.34 |
| 21 | 1.44 | −2.38 |
| 25 | 0.93 | −2.89 |

TABLE 3b
mean counts of propionibacteria

| Days of | Mean log | Change from |
|---|---|---|
| application | count/cm² skin | baseline |
| 0 (baseline) | 2.78 | n/a |
| 6 | 3.24 | +0.46 |
| 8 | 2.46 | −0.32 |
| 11 | 2.25 | −0.53 |
| 21 | 2.46 [1.5]* | −0.32 [−1.28]* |
| 25 | 1.41 | −1.37 |

*Figures in brackets omit spurious data point from one sample site.

The samples collected two weeks after the end of the treatment programme showed a greater than 2 log reduction in numbers of coagulase-negative staphylococci with respect to the baseline. Numbers of propionibacteria had returned to pre-treatment values.

It can be seen from these data that the formulation of the invention is effective in vivo as an antimicrobial agent against coagulase-negative staphylococci (Table 3a). It is also effective in vivo against propionibacteria (Table 3b). It appears to be reaching the targeted follicles and once there, to be successfully acting against the resident bacteria, significantly reducing the numbers of bacteria present at the skin surface following treatment. The formulation is therefore suitable for use as a topical antimicrobial agent, and as a topical anti-acne treatment.

Moreover the antibacterial effect appears to be sustained for a significant period of time following each topical application, making the formulation suitable for use as a “leave-on” skin treatment product. The data demonstrate the substantivity of the formulation, suggesting that the copper usnate remains in the stratum corneum, where it can continue to exert an antimicrobial effect, for some time after application to the skin. This would be consistent with delivery of the active to the follicles via the stratum corneum.

### Example 4

### Antimicrobial Activity In Vivo (Formulation B)

Example 3 was repeated, but using formulation B as described in Example 2. In this case samples were taken before the treatment began, and at 10, 11 and 14 days afterwards whilst the treatment continued. No data were collected after the treatment programme had finished.

The results are shown in Tables 4a and 4b below, for coagulase-negative staphylococci and propionibacteria respectively.

TABLE 4a
mean counts of coagulase-negative staphylococci

| Days of | Mean log | Change from |
|---|---|---|
| application | count/cm² skin | baseline |
| 0 (baseline) | 4.91 | n/a |
| 10 | 2.72 | −2.19 |
| 11 | 3.07 | −1.84 |
| 14 | 2.98 | −1.93 |

TABLE 4b
mean counts of propionibacteria

| Days of | Mean log | Change from |
|---|---|---|
| application | count/cm² skin | baseline |
| 0 (baseline) | 6.35 | n/a |
| 10 | 5.04 | −1.31 |
| 11 | 5.05 | −1.30 |
| 14 | 3.68 [5.22]* | −2.67 [−1.13]* |

*Figures in brackets omit spurious (inconsistent) data point from one sample site.

It can be seen from the Table 4 data that formulation B is also effective in vivo as an antimicrobial agent against both coagulase-negative staphylococci and propionibacteria, and moreover that it appears to be reaching bacteria present in the pilosebaceous follicles. It is therefore suitable for use as a topical antimicrobial agent, and as a topical anti-acne treatment. As in Example 3, the formulation appears to be providing a sustained antibacterial effect following each application.

A formulation according to the present invention may for instance be used either to treat, or to help prevent, acne or another bacterial infection associated with the skin. As an anti-acne agent it may be administered topically to acne-affected skin, in particular as a “leave-on” treatment, and can provide sustained efficacy of the usnic acid or usnate active for several hours after application. The formulation can therefore be used as part of a daily (and/or nightly) skin treatment regime.

Usnic acid and usnate salts such as copper usnate are also known to be active against other micro-organisms which reside in the stratum corneum or the follicles, for example staphylococci such as S. aureus. Copper usnate, for example, has been found by the present inventors to have an MIC against S. aureus ATCC 29213 of 7.8 μg/ml, and an MBC against the organism of 62.5 μg/ml; it has also been found to have an MIC against Corynebacterium striatum NCTC 764 of 3.9 μg/ml, and an MBC against that organism of 62.5 μg/ml. Thus, a formulation according to the invention may be for use as a topical antimicrobial (in particular antibacterial) formulation against one or more such micro-organisms. It may for example be applied as a skin cleanser or disinfectant, or as a hand or face or body wash, or as a deodorant.

### Example 5

### Formulations C to J

Further skin treatment formulations C to J were prepared using the components and concentrations listed in Table 5. Concentrations are quoted as percentages by weight. Formulations C to F, which contained no thickener, had the form of solutions rather than gels.

TABLE 5

| Ingredient | C | D | E | F | G | H | J |
|---|---|---|---|---|---|---|---|
| Copper usnate | 1 | 1 | 1 | 1 | 0.96 | 1 | 1 |
| DMI | 30 | 30 | 40 | 30 | 28.7 | 30 | 29.95 |
| Homosalate | 10 | 10 | 10 | 10 | 9.6 | 10 | 9.98 |
| Glyceryl | 20 | 38 | 19 | 39 | | | |
| diisostearate | | | | | | | |
| PCA glyceryl | | | | | 4.8 | 5 | 4.99 |
| oleate | | | | | | | |
| THFA | 20 | | 20 | | | | |
| Ethanol | | 19 | | 20 | | | |
| Labrasol ™ | 18 | | 10 | | 9.6 | 10 | 10 |
| Ethyl | | | | | 19.1 | 20 | 19.97 |
| pyrrolidone | | | | | | | |
| Methyl | | | | | 4.8 | 5 | 4.99 |
| glucose | | | | | | | |
| dioleate | | | | | | | |
| Ethyl pyruvate | | | | | 19.1 | 14.5 | 14.55 |
| Oleic acid | | | | | 0.49 | 0.5 | 0.57 |
| PCA | | 1 | | | | | |
| Antioxidant | | | | | 0.95 | 1 | 1 |
| Thickener | | | | | 1.9 | 2 | 2 |
| Salicylic acid | 1 | 1 | | | | 1 | 1 |
| Total | 100 | 100 | 100 | 100 | 100 | 100 | 100 |

The THFA, ethanol and salicylic acid were sourced from Sigma-Aldrich, UK, and the glyceryl diisostearate from Dr Straetmans Chemische Produkte GmbH.

The formulations were prepared using an analogous method to that of Examples 1 and 2. The copper usnate was premixed with the DMI, the homosalate and where appropriate the ethyl pyrollidone, THFA and ethyl pyruvate. The mixture was heated and stirred to optimise usnate dissolution, then filtered to remove any undissolved residue. The glyceryl fatty acid ester was then added, along with the remaining components of the solvent system, and mixed vigorously. To this mixture was added, as appropriate, the PCA, salicylic acid and antioxidant, followed by the thickener. The resultant mixture was stirred and heated, shaken vigorously and if necessary left to gel at room temperature.

### Example 6

### Antimicrobial Activity In Vivo (Formulations C to J)

These experiments used formulations C to J as described in Example 5, containing copper usnate as an antimicrobial and anti-acne active substance.

The effects of the formulations on the surface populations of coagulase-negative staphylococci and propionibacteria were tested on single individuals, using similar treatment regimens and sampling techniques to those of Example 3 (although the times at which the skin was sampled varied from case to case). Samples were taken from three locations on the subject's face: the forehead (FH), left cheek (LC) and right cheek (RC).

The results are shown in Tables 6a to 6g below, for formulations C to J respectively.

TABLE 6a
(formulation C)

| | | | | | |
|---|---|---|---|---|---|
| Days of | | | | | Change from |
| application | FH | LC | RC | Mean | baseline |

Coagulase-negative
staphylococci
log count/cm² skin

| | | | | | |
|---|---|---|---|---|---|
| 0 (baseline) | 2.67 | 4.09 | 4.80 | 3.85 | n/a |
| 7 | 2.26 | 2.40 | 2.31 | 2.32 | −1.53 |

Propionibacteria
log count/cm² skin

| | | | | | |
|---|---|---|---|---|---|
| 0 (baseline) | 5.53 | 5.61 | 6.28 | 5.81 | n/a |
| 7 | 4.78 | 5.09 | 3.52 | 4.46 | −1.35 |

TABLE 6b
(formulation D)

| | | | | | |
|---|---|---|---|---|---|
| Days of | | | | | Change from |
| application | FH | LC | RC | Mean | baseline |

Coagulase-negative
staphylococci
log count/cm² skin

| | | | | | |
|---|---|---|---|---|---|
| 0 (baseline) | 5.68 | 4.04 | 4.67 | 4.9 | n/a |
| | 5.73 | 4.40 | 4.75 | | |
| 5 | 2.31 | 3.13 | 3.12 | 2.85 | −2.05 |

Propionibacteria
log count/cm² skin

| | | | | | |
|---|---|---|---|---|---|
| 0 (baseline) | 6.62 | 6.18 | 6.34 | 6.38 | n/a |
| | 6.56 | 6.12 | 6.43 | | |
| 5 | 5.77 | 5.75 | 6.15 | 5.89 | −0.48 |

TABLE 6c
(formulation E)

| | | | | | |
|---|---|---|---|---|---|
| Days of | | | | | Change from |
| application | FH | LC | RC | Mean | baseline |

Coagulase-negative
staphylococci
log count/cm² skin

| | | | | | |
|---|---|---|---|---|---|
| 0 (baseline) | 5.50 | 4.82 | 4.85 | 5.15 | n/a |
| | 5.71 | 4.88 | 5.12 | | |
| 1 | 4.66 | 4.19 | 4.33 | 4.39 | −0.75 |
| 6 | 3.13 | 3.50 | 3.84 | 3.49 | −1.66 |
| 8 | 3.61 | 3.70 | 3.72 | 3.68 | −1.47 |
| 12 | 4.21 | 3.67 | 3.60 | 3.83 | −1.32 |
| 14 | 3.56 | 3.31 | 3.27 | 3.38 | −1.77 |

Propionibacteria
log count/cm² skin

| | | | | | |
|---|---|---|---|---|---|
| 0 (baseline) | 6.10 | 6.50 | 6.59 | 6.50 | n/a |
| | 6.72 | 6.56 | 6.61 | | |
| 1 | 6.52 | 6.31 | 6.42 | 6.42 | −0.09 |
| 6 | 5.41 | 6.15 | 6.33 | 5.96 | −0.54 |
| 8 | 5.60 | 5.70 | 5.78 | 5.69 | −0.81 |
| 12 | 5.93 | 5.80 | 5.66 | 5.80 | −0.71 |
| 14 | 5.48 | 4.78 | 5.46 | 5.24 | −1.26 |

TABLE 6d
(formulation F)

| | | | | | |
|---|---|---|---|---|---|
| Days of | | | | | Change from |
| application | FH | LC | RC | Mean | baseline |

Coagulase-negative
staphylococci
log count/cm² skin

| | | | | | |
|---|---|---|---|---|---|
| 0 (baseline) | 2.15 | 4.72 | 4.36 | 3.74 | n/a |
| 6 | 2.18 | 3.15 | 3.47 | 2.93 | −0.81 |

Propionibacteria
log count/cm² skin

| | | | | | |
|---|---|---|---|---|---|
| 0 (baseline) | 4.77 | 6.14 | 5.80 | 5.57 | n/a |
| 6 | 4.72 | 5.25 | 4.89 | 4.95 | −0.62 |

TABLE 6e
(formulation G)

| | | | | | |
|---|---|---|---|---|---|
| Days of | | | | | Change from |
| application | FH | LC | RC | Mean | baseline |

Coagulase-negative
staphylococci
log count/cm² skin

| | | | | | |
|---|---|---|---|---|---|
| 0 (baseline) | 4.67 | 4.87 | 5.18 | 4.91 | n/a |
| 10 | 3.10 | 2.33 | 2.73 | 2.72 | −2.19 |
| 11 | 2.44 | 3.50 | 3.26 | 3.07 | −1.84 |
| 14 | 2.66 | 3.41 | 2.86 | 2.98 | −1.93 |

Propionibacteria
log count/cm² skin

| | | | | | |
|---|---|---|---|---|---|
| 0 (baseline) | 6.09 | 6.41 | 6.55 | 6.35 | n/a |
| 10 | 4.35 | 5.52 | 5.25 | 5.04 | −1.31 |
| 11 | 4.35 | 5.60 | 5.19 | 5.05 | −1.30 |
| 14 | 0.61 | 5.59 | 4.95 | 3.72 | −2.63 |

TABLE 6f
(formulation H)

| | | | | | |
|---|---|---|---|---|---|
| Days of | | | | | Change from |
| application | FH | LC | RC | Mean | baseline |

Coagulase-negative
staphylococci
log count/cm² skin

| | | | | | |
|---|---|---|---|---|---|
| 0 (baseline) | 5.10 | 5.14 | 4.24 | 4.76 | n/a |
| | 5.20 | 4.64 | 4.22 | | |
| 3 | 1.61 | 3.54 | 1.99 | 2.38 | −2.38 |
| 7 | 2.02 | 2.33 | 1.79 | 2.05 | −2.71 |
| 10 | 1.21 | 2.29 | 2.14 | 1.88 | −2.88 |
| 11 | 1.21 | 2.18 | 2.93 | 2.11 | −2.65 |
| 14 | 2.33 | 2.53 | 3.21 | 2.69 | −2.07 |

Propionibacteria
log count/cm² skin

| | | | | | |
|---|---|---|---|---|---|
| 0 (baseline) | 6.19 | 6.33 | 6.70 | 6.45 | n/a |
| | 6.53 | 6.43 | 6.49 | | |
| 3 | 4.33 | 5.75 | 5.46 | 5.18 | −1.27 |
| 7 | 4.97 | 5.65 | 5.48 | 5.37 | −1.08 |
| 10 | 3.86 | 5.50 | 5.05 | 4.80 | −1.64 |
| 11 | 4.66 | 5.53 | 4.97 | 5.05 | −1.39 |
| 14 | 3.93 | 5.58 | 5.33 | 4.95 | −1.50 |

TABLE 6g
(formulation J)

| | | | | | |
|---|---|---|---|---|---|
| Days of | | | | | Change from |
| application | FH | LC | RC | Mean | baseline |

Coagulase-negative
staphylococci
log count/cm² skin

| | | | | | |
|---|---|---|---|---|---|
| 0 | 1.51 | 4.46 | 4.15 | 3.37 | n/a |
| 7 | 0.91 | 2.34 | 1.89 | 1.71 | −1.66 |
| 12 | 2.55 | 2.72 | 2.71 | 2.66 | −0.71 |
| 20 | 1.31 | 2.21 | 2.68 | 2.07 | −1.31 |

Propionibacteria
log count/cm² skin

| | | | | | |
|---|---|---|---|---|---|
| 0 | 5.09 | 6.13 | 6.12 | 5.78 | n/a |
| 7 | 2.23 | 5.66 | 3.37 | 3.75 | −2.03 |
| 12 | 2.49 | 5.34 | 4.57 | 4.13 | −1.65 |
| 20 | 2.21 | 5.81 | 5.78 | 4.60 | −1.18 |

In all cases, the invented formulations caused a marked reduction in numbers of both coagulase-negative staphylococci and propionibacteria compared to the pre-treatment values. These data demonstrate that a formulation containing an usnate salt solubilised in the components (i) to (iii) can effectively deliver the usnate to the skin, including to bacteria present in the follicles. Such a formulation is therefore suitable for use as a topical antimicrobial agent, and as a topical anti-acne treatment.

### Example 7

### Antimicrobial Activity In Vivo (Formulation H)

Six human volunteers were treated for 14 days using formulation H from Example 5, using similar treatment regimens and sampling techniques as described in Example 3.

Surface propionibacteria and staphylococci were sampled using the scrub method of Williamson and Kligman, again as described in Example 3. Follicular propionibacteria and staphylococci were also enumerated using the cyanoacrylate glue technique (Holland K T, Roberts C D 1974, “A technique for sampling micro-organisms from the pilo-sebaceous ducts”, J Appl Bacteriol 37(3): 289-96).

The results are shown in Tables 7 and 8 below, for the surface and follicular bacteria respectively.

TABLE 7

| Days of | | Change from | |
|---|---|---|---|
| application | | baseline | **P value |
| | *Coagulase-negative | | |
| | staphylococci | | |
| | Mean log count/cm² skin | | |
| 0 (baseline) | 3.9 | n/a | n/a |
| 1 | 2.9 | −1.0 | <0.0001 |
| 4 | 2.3 | −1.6 | <0.0001 |
| 7 | 2.5 | −1.4 | <0.0001 |
| 10 | 2.2 | −1.7 | <0.0001 |
| 14 | 2.4 | −1.5 | <0.0001 |
| | Propionibacteria | | |
| | Mean log count/cm² skin | | |
| 0 (baseline) | 5.6 | n/a | n/a |
| 1 | 4.9 | −0.7 | 0.0002 |
| 4 | 4.5 | −1.1 | <0.0001 |
| 7 | 4.5 | −1.1 | <0.0001 |
| 10 | 4.5 | −1.1 | <0.0001 |
| 14 | 4.6 | −1.0 | 0.0007 |

*Mean values were calculated from the data obtained from six volunteers sampled at three sites: forehead, right cheek and left cheek.
**A statistical analysis was carried out using a paired Student's t test. Significance is indicated by P ≦ 0.05.

TABLE 8

| Days of | | Change from | |
|---|---|---|---|
| application | | baseline | **P value |
| | *Coagulase-negative | | |
| | staphylococci | | |
| | Mean log count/cm² skin | | |
| 0 (baseline) | 3.7 | n/a | n/a |
| 14 | 0.5 | −3.2 | 0.001 |
| | Propionibacteria | | |
| | Mean log count/cm² skin | | |
| 0 (baseline) | 5.3 | n/a | n/a |
| 14 | 3.9 | −1.4 | 0.1 |

*Mean values were calculated from the data obtained from six volunteers sampled at three sites from the central forehead.
**A statistical analysis was carried out using a paired Student's t test. Significance is indicated by P ≦ 0.05.

Table 7 shows that formulation H significantly reduced the numbers of coagulase-negative staphylococci and propionibacteria at the skin surface of this treatment cohort. Table 8 shows that the formulation also produced a highly significant reduction in the follicular staphylococci and a mean log cm⁻² reduction in follicular propionibacteria of 1.4. Thus, again, the formulation has been demonstrated to be capable of exerting antimicrobial activity within the pilosebaceous follicles.

### Example 8

### Formulations K to N

Further skin treatment gel formulations K to N were prepared using the components and concentrations listed in Table 9. Concentrations are quoted as percentages by weight.

Two corresponding “placebo” formulations, KP and LP, were also prepared, again as shown in Table 9. These were based on formulations K and L respectively, but contained no copper usnate.

TABLE 9

| Ingredient | K | L | M | N | KP | LP |
|---|---|---|---|---|---|---|
| Copper usnate | 1.00 | 1.00 | 1.00 | 1.00 | | |
| DMI | 30.00 | 30.00 | 30.00 | 30.00 | 30.00 | 30.00 |
| Homosalate | 8.50 | 8.50 | 8.50 | 7.00 | 8.50 | 8.50 |
| Glyceryl | | 5.00 | 5.00 | 5.00 | | 5.00 |
| diisostearate | | | | | | |
| PCA glyceryl | 5.00 | | 5.00 | | 5.00 | |
| oleate | | | | | | |
| THFA | 10.00 | 15.00 | 10.00 | 15.00 | 10.00 | 15.00 |
| Ethanol | 10.00 | 5.00 | 10.00 | 5.00 | 10.00 | 5.00 |
| Labrasol ™ | 20.00 | 25.00 | 15.00 | 22.00 | 21.00 | 26.00 |
| Methyl glucose | 5.00 | 5.00 | 5.00 | 5.00 | 5.00 | 5.00 |
| dioleate | | | | | | |
| Hydroxypropyl | 2.50 | 2.50 | 2.50 | | 2.50 | 2.50 |
| cellulose | | | | | | |
| (GF Pharm) | | | | | | |
| Carbopol ™ | | | | 7.00 | | |
| Ultrez 10 | | | | | | |
| Salicylic acid | 1.00 | 1.00 | 1.00 | 1.00 | 1.00 | 1.00 |
| PEG 400 | 7.00 | 2.00 | 7.00 | 2.00 | 7.00 | 2.00 |
| Total | 100.00 | 100.00 | 100.00 | 100.00 | 100.00 | 100.00 |

The DMI was sourced as Arlasolve DMI from Croda, the THFA from Pennakem, the Labrasol™ from Gattefosse, the homosalate from Symrise, the methyl glucose dioleate from Lubrizol, the PCA glyceryl oleate as DermoFeel™ P-30 from Dr Straetmans Chemische Produkte GmbH, the glyceryl diisostearate from Stearinierer Dubois, the salicylic acid (BP grade) from A & E Cannock, the hydroxypropyl cellulose thickener as GF Pharm (molecular weight 370,000) from Ashland, the Carbopol™ gelling agent from Lubrizol, the ethanol from Merck or Fisher Scientific and the PEG 400 from Merck. The copper usnate was sourced from Onyx Scientific Ltd, UK.

Formulations K to N were prepared by loading the copper usnate into a clean, dry vessel, and then adding, stepwise, the ingredients (a) DMI, (b) homosalate, (c) THFA, (d) ethanol, (e) PEG 400, (f) Labrasol™ and (g) salicylic acid. The resultant mixture was protected from light using aluminium foil, placed in a water bath at 50° C. and stirred until the copper usnate was fully dissolved. The PCA glyceryl oleate or glyceryl diisostearate was then added, and also the methyl glucose dioleate, and again this mixture was protected from light and stirred in a water bath at 50° C.

The mixture was then removed from the water bath and cooled to ambient temperature. The thickener (hydroxypropyl cellulose or Carbopol™) was added gradually, whilst homogenising the mixture, until evenly dispersed. The resultant formulation was then stirred at room temperature for at least 16 hours to ensure solvation of the copper usnate and salicylic acid.

Formulations KP and LP were prepared in an analogous fashion, but omitting the copper usnate.

These gel formulations were found to have an acceptable appearance and odour, an even consistency and a pleasing, non-greasy, feel when applied to the skin. They were also absorbed fairly quickly into the skin and were easy to apply. Formulation L performed particularly well in terms of skin feel, formulation K in terms of odour. In all cases, the copper usnate active was completely dissolved in the solvent system at 1% w/w.

Examples 9 to 12 below also demonstrate that the formulations had good physical and chemical stability.

### Example 9

### Stability Tests i (Formulations K and L)

Samples of formulations K and L were stored at 40° C. and 75% RH for 12 weeks. At certain time points, the samples were assessed for copper usnate content and purity. The assessments were carried out by HPLC, using 0.1% v/v aqueous acetic acid as the mobile phase A, acetonitrile as the mobile phase B and diethylene glycol monoethyl ether (99%, Transcutol™ P, ex Gattefosse) as the standard and sample diluent.

Table 10 (appended) shows the results, in terms of the percentage of copper usnate recovered from the samples at each time point, and its peak purity. Values at t=0 represent the mean value±SD, n=6. Values at other time points represent the mean value±range, n=3.

It can be seen from Table 10 that even after 12 weeks' storage at elevated temperatures, the level of copper usnate remaining within each formulation exceeded 0.93% w/w. After 12 weeks formulation L showed a marginally lower level of copper usnate in comparison to its value at t=0. For formulation K, however, only a negligible difference was observed between the t=0 and t=12 copper usnate percentages.

### Example 10

### Stability Tests ii (Formulations K, L, KP and LP)

Samples of formulations K, L, KP and LP were stored at 40° C. and 75% RH for 12 weeks. At certain time points, the samples were assessed for changes in their viscosity, using a Brookfield™ LV-DV-1+ viscometer with Spindle 25 at 12 rpm. The viscosities of formulations L and LP were also assessed using Spindle 34 at 6 rpm, due to a low torque value with Spindle 25 All viscosities were determined at 25° C.

The results are shown in Table 11 (appended). It can be seen that formulation K and its placebo equivalent KP showed no notable change in viscosity during the storage period. Formulations L and LP performed marginally less well, exhibiting a small reduction in viscosity and also demonstrating a slight inconsistency in texture during the tests.

### Example 11

### Stability Tests iii (Formulations K, L, KP and LP)

Samples of formulations K, L, KP and LP were stored at 40° C. and 75% RH for 12 weeks. At certain time points, the samples were assessed for changes in their macroscopic and microscopic appearance. Macroscopic appearance was judged by eye, primarily to detect changes in physical stability and phase separation. Colour was assessed using the Pantone Formula Guide. Microscopic observations were made by light microscopy, with magnifications of 200× and 400×, and using both polarised and non-polarised light, in order to detect the emergence of precipitated copper usnate crystals.

The results are summarised in appended Table 12. During the storage period there were no noticeable differences in the colours of any of the four formulations, and no microscopic evidence of copper usnate crystals. Formulation K performed particularly well, showing no evidence of a change in its physical stability over the full 12 week stability period. In formulations L and LP, there was some increase in agglomeration during storage. It is possible that this might be due to the higher concentration of THFA in formulation L, in combination with the other excipients present, leading to less consistent gelling properties, although we do not wish to be bound by this theory.

### Example 12

### Stability Tests iv (Formulations K, L, KP and LP)

The formulation samples used in Examples 9 to 11 were stored in 15 mL polypropylene airless pump dispensers, such as might typically be used to store skin treatment formulations. These pumps were assessed visually, and also weighed in order to detect potential weight loss, at intervals during the 12 week stability test period. No noticeable weight changes were observed, and no changes in the internal appearance of the pumps when in contact with any of the test formulations.

Thus, the results from Examples 9 to 12 indicate the stability of formulations according to the invention, their suitability as vehicles for the antimicrobial agent copper usnate, and their potential use for the topical treatment of the skin.

### Example 13

### In Vitro Antimicrobial Activity (Formulations K and L)

Samples of formulations K and L, along with pure copper usnate (Onyx Scientific Ltd, UK) solubilised in DMSO, were tested for their in vitro antimicrobial activity.

The test organism used was Propionibacterium acnes NCTC 737. This is a propionibacterial strain and is the type strain of the genus; it is fully susceptible to antibiotics. The propionibacteria are clinically significant due to their involvement in acne. They are also opportunistic pathogens in compromised hosts. Activity observed against these micro-organisms is expected to be a good predictor of activity against acne.

The propionibacteria were cultured and maintained on Wilkins-Chalgren Anaerobe Medium (agar and broth) at pH 6.0; all cultures were incubated anaerobically at 37° C. for 72 hours.

The following tests were carried out to assess antimicrobial activity against the test organism.

(a) Minimum Inhibitory Concentration (MIC) Assay

This is a standard international method for quantitatively assessing the antimicrobial activity of a compound/formulation in a liquid medium. The method used a sterile 96-well microtitre plate, capable of holding about 200 μl of liquid per well. The wells contained liquid culture medium and ranges of decreasing concentrations of the relevant test compound/formulation (in this case reported as the relative concentration of the active molecule—copper usnate—in the formulations) in doubling dilutions (eg 1000, 500, 250, 125 . . . μg/ml, etc, down to 0.49 μg/ml). The culture media were as described above.

The wells were inoculated with a liquid suspension of freshly grown micro-organism and incubated under the conditions described above. After incubation, the microtitre plate was examined visually for cloudiness in each well, which would indicate microbial growth. The MIC value was recorded as the lowest concentration of test compound/formulation required to inhibit microbial growth, ie the lowest concentration for which the liquid in the well remained clear.

The assays included both negative (culture medium with no micro-organisms) and positive (culture medium plus diluting solvent plus micro-organism) controls.

Since inhibition does not necessarily indicate killing of microbial cells, merely that growth as visible to the naked eye has been inhibited, it is desirable to conduct a further test (the MBC assay described below) to establish the concentration of the test compound/formulation needed to kill the test organism.

(b) Minimum Biocidal Concentration (MBC) Assay

This assay, normally carried out after an MIC assay, determines the minimum concentration of a compound or formulation that is lethal to the test micro-organism.

Following an MIC assay, a 5 μl sample was withdrawn from the first microtitre well that showed positive growth and from all the subsequent wells that showed no growth. These samples were then individually sub-cultured on antibiotic-free agar medium, under the incubation conditions described above. Following incubation they were examined visually for microbial growth. The MBC was taken to be the lowest test compound/formulation concentration (in this case reported as the relative concentration of copper usnate for the formulations) for which the incubated sample showed no growth.

The ratio of MIC to MBC should ideally be as close to 1 as possible. This facilitates selection of the lowest possible effective concentration of a test compound or formulation, with a reduced risk of selecting a sub-lethal concentration which could promote resistance or allow the target microbial population to recover.

(c) Synthetic Sebum Time-to-Kill Assay

This quantitative assay was designed to assess the level of kill of a P. acnes culture in a synthetic sebum (non-aqueous environment) over a defined time period, in this case 4 hours.

A culture of P. acnes NCTC 737 was inoculated into a synthetic sebum (a liquid mixture of lipid components designed to simulate human sebum) containing the relevant test compound or formulation diluted in 1-octanol. From this culture, samples were taken after 4 hours, 10-fold serially diluted in wash fluid (0.075 M sodium phosphate buffer, pH 7.9, 0.1% v/v Triton™-X 100) and inoculated onto agar plates (in triplicate). The plates were then incubated as described above and subsequently examined visually for growth. The numbers of viable microbial colonies on the plates were counted with the aid of a colony counter and converted to colony-forming units per ml (cfu/ml) using the appropriate dilution factor. Cell counts conducted in the absence of formulations (but still in the presence of 1-octanol) acted as positive controls.

All experiments were conducted in triplicate.

The MIC and MBC results are shown in Table 13 below and the synthetic sebum time-to-kill results in Table 14.

TABLE 13

| | MIC | MBC | MIC:MBC |
|---|---|---|---|
| Compound/formulation | (μg/ml) | (μg/ml) | ratio |
| Copper usnate (Onyx) | 0.24 | 0.98 | 0.25 |
| Formulation K (Cu | <0.12 | 0.98 | <0.125 |
| usnate equivalent) | | | |
| Formulation L (Cu | <0.12 | 0.49 | <0.25 |
| usnate equivalent) | | | |

TABLE 14

| | Log10 cfu/ml | Log10 cfu/ml | Log10 |
|---|---|---|---|
| Compound/formulation | (T0 hours) | (T4 hours) | change |
| Control (1-octanol) | 7.97 | 8.1 | 0.13 |
| Copper usnate 49 μg/mL | 7.97 | 3.0* | −4.97 |
| (Onyx) | | | |
| Formulation K (49 μg/mL Cu | 7.97 | 3.0* | −4.97 |
| usnate equivalent) | | | |
| Formulation L (49 μg/mL Cu | 7.97 | 3.0* | −4.97 |
| usnate equivalent) | | | |

*Lowest detectable level = 3.0 Log10 cfu/ml

The data in Tables 13 and 14 show that both the invented formulations are antibacterially active against P. acnes NCTC 737, and thus likely to be of use as anti-acne agents. Moreover, in the MIC tests they appeared to be slightly more active (in terms of relative copper usnate levels) compared to the unformulated copper usnate.

At the concentrations tested, the invented formulations also demonstrated an excellent level of antimicrobial activity against P. acnes NCTC 737 in a lipid environment which mimics that of human skin. This retention of activity in the presence of lipid further indicates the utility of the invented formulations as topical anti-acne agents.

### Example 14

### Skin Patch Tests (Formulations K and L)

Samples of each of the formulations K and L were applied to the skin of three human volunteers using 8 mm Finn chamber patch test units. After 24 hours the test strips were carefully removed and the results read. Each patch test was scored according to the reaction seen on the skin, using the grading system of Wilkinson et al (Wilkinson D S, Fregert S, Magnusson B, Bandmann H J, Calnan C D, Cronin E, Hjorth N, Maibach H J, Malalten K E, Meneghini C L, Pirilä V, “Terminology of contact dermatitis”, Acta Derm Venereol (1970), 50(4): 287-92). This grading system is summarised in Table 15 below.

TABLE 15

| Score | Interpretation |
|---|---|
| − | Negative reaction |
| ?+ | Doubtful reaction, faint erythema only |
| + | Weak (nonvesicular) reaction, erythema, slight |
| | infiltration |
| ++ | Strong (edematous or vesicular) reaction; erythema, |
| | infiltration, vesicles |
| +++ | Extreme reaction (bullous and ulcerative) |
| IR | Irritant reactions of different types |
| NT | Not tested |

Both test formulations produced a negative reaction in all three of the volunteers, ie no indication of skin irritancy. This confirms their suitability for use as topical skin treatment formulations.

### Example 15

### Sensory Evaluation (Formulations K and L)

Seven human volunteers were asked to apply small amounts of a test formulation to a section of their forearm. They were asked to rub in the formulation as they would any other cosmetic product, noting how the formulation looked, felt and behaved on and after application. The volunteers were then asked to complete a questionnaire relating to the sensory profile of the formulation. This process was repeated until each volunteer had evaluated seven test formulations, which included both formulations K and L and also a commercially available topical spot treatment cream. The different formulations were randomly assigned for application to different sections of the forearm for each volunteer, and the volunteers did not know the identities or contents of the formulations they were applying.

When asked initially to rate the appearance of the formulations, none of the volunteers expressed dislike for formulations K and L. 14.3% of the volunteers liked both the formulations a lot. As regards the colour of the formulations, none of the volunteers expressed dislike for formulation K, although 14.3% did express a slight dislike for the colour of formulation L. As regards the consistency of the formulations, 100% of the volunteers assessed formulation L as being “about right” (ie neither too thick nor too thin), and 71.4% assessed formulation K as being “about right”.

The volunteers were then asked how pleasant or unpleasant the formulation felt on application to the skin. 100% of them regarded formulation K as either “very pleasant”, “fairly pleasant” or “neither pleasant nor unpleasant”: 85.8% categorised formulation L in this way. As regards the speed with which the formulations were perceived to absorb into the skin, 57.2% of the volunteers categorised formulation K as absorbing “very quickly”, “fairly quickly” or “neither quickly nor slowly”, whilst 100% categorised formulation L in this way (85.7% believing that formulation L was absorbed “fairly quickly”).

None of the volunteers categorised either of the formulations K and L as being difficult to spread on the skin: in fact, 100% of them regarded formulation K as being either “very easy” or “fairly easy” to spread, whilst 85.8% of them categorised formulation L in this way and the remainder regarded formulation L as “neither easy nor difficult” to spread.

After application of the test formulation to the skin, 57.1% of the volunteers believed that formulation L left their skin feeling “neither greasy nor dry” (as compared to 42.9% feeling the same way about the commercially available product). For formulation K, 85.8% of the volunteers said that their skin felt either “fairly greasy”, “fairly dry” or “neither greasy nor dry”.

These results indicate that the formulations according to the invention are generally acceptable in terms of their sensory performance, for application to human skin. Not only are they chemically and physically stable (as demonstrated for instance in Examples 9 to 12), and antimicrobially active (Example 13), but they also look and feel appropriate to consumers and apply well to the skin, without (Example 14) causing irritation.

TABLE 10

| | Formulation L | | Formulation K | |
|---|---|---|---|---|
| Stability | % w/w of | | % w/w of | |
| time point | copper usnate | Peak purity | copper usnate | Peak purity |
| t = 0 | 0.9936 ± 0.0128 | 98.18 ± 0.06 | 0.9762 ± 0.0127 | 98.20 ± 0.19 |
| t = 2 weeks | 0.9686 ± 0.0083 | 98.29 ± 0.02 | 0.9674 ± 0.0241 | 98.03 ± 0.08 |
| t = 4 weeks | 0.9746 ± 0.0027 | 97.95 ± 0.21 | 0.9798 ± 0.0217 | 98.05 ± 0.19 |
| t = 8 weeks | 0.9534 ± 0.0041 | 97.85 ± 0.09 | 0.9684 ± 0.0077 | 98.01 ± 0.06 |
| t = 12 weeks | 0.9387 ± 0.0245 | 97.02 ± 0.06 | 0.9652 ± 0.0169 | 97.42 ± 0.44 |

TABLE 11

| | | | t = 0 | | t = 2 weeks* | | t = 4 weeks* | |
|---|---|---|---|---|---|---|---|---|
| | Conditions | | Viscosity | Torque | Viscosity | Torque | Viscosity | Torque |
| Formulation | Spindle | RPM | (cP) | (%) | (cP) | (%) | (cP) | (%) |
| L | S34 | 6 | 4030 | 40.2 | 4012 | 40.1 | 3660 | 36.6 |
| | S25 | 12 | 3160 | 7.9 | 3160 | 7.9 | 2720 | 6.8 |
| LP | S34 | 6 | 3260 | 32.6 | 2440 | 24.4 | 2310 | 23.1 |
| | S25 | 12 | 2840 | 7.1 | 2560 | 6.9 | 2280 | 5.7 |
| K | S25 | 12 | 34000 | 85.0 | 33800 | 86.7 | 35400 | 88.5 |
| KP | S25 | 12 | 16000 | 40.0 | 16720 | 41.7 | 16200 | 40.5 |

| | | | t = 8 weeks* | | t = 12 weeks* | |
|---|---|---|---|---|---|---|
| | Conditions | | Viscosity | Torque | Viscosity | Torque |
| Formulation | Spindle | RPM | (cP) | (%) | (cP) | (%) |
| L | S34 | 6 | 3150 | 31.5 | 3320 | 33.2 |
| | S25 | 12 | 3000 | 7.4 | 3200 | 8.0 |
| LP | S34 | 6 | 1450 | 14.5 | 2430 | 24.3 |
| | S25 | 12 | 1240 | 3.1 | 2400 | 6.0 |
| K | S25 | 12 | 36160 | 90.4 | 35280 | 88.2 |
| KP | S25 | 12 | 18800 | 46.7 | 15680 | 39.1 |

*t = 2, t = 4, t = 8 and t = 12 week stability samples stored at 40° C.

TABLE 12

| | t = 0 | | t = 2 weeks | | t = 4 weeks | |
|---|---|---|---|---|---|---|
| | Macroscopic | Microscopic | Macroscopic | Microscopic | Macroscopic | Microscopic |
| Formulation | appearance | appearance | appearance | appearance | appearance | appearance |
| L | Dark green clear gel | No evidence of | Appearance of | No evidence of | Small agglomerates | No evidence of |
| | (PFG 327C)* | copper usnate | agglomerates in a | copper usnate | observed in a dark | copper usnate |
| | | crystals | dark green clear gel | crystals | green clear gel (PFG | crystals |
| | | | (PFG 327C)* | | 327C)* | |
| K | Dark green clear gel | No evidence of | Dark green clear gel | No evidence of | Dark green clear gel | No evidence of |
| | (PFG 327C)* | copper usnate | (PFG 327C)* | copper usnate | (PFG 327C)* | copper usnate |
| | | crystals | | crystals | | crystals |
| LP | Pale yellow clear gel | n/a | Appearance of small | n/a | Small agglomerates | n/a |
| | (PFG 1205C)* | | agglomerates in a | | observed in a pale | |
| | | | pale yellow clear gel | | yellow clear gel (PFG | |
| | | | (PFG 1205C)* | | 1205C)* | |
| KP | Pale yellow clear gel | n/a | Pale yellow clear gel | n/a | Pale yellow clear gel | n/a |
| | (PFG 1205C)* | | (PFG 1205C)* | | (PFG 1205C)* | |

| | t = 8 weeks | | t = 12 weeks | |
|---|---|---|---|---|
| | Macroscopic | Microscopic | Macroscopic | Microscopic |
| Formulation | appearance | appearance | appearance | appearance |
| L | Small-medium | No evidence of | Small-medium | No evidence of |
| | agglomerates observed | copper usnate | agglomerates observed | copper usnate |
| | in a dark green clear | crystals | in a dark green clear | crystals |
| | gel (PFG 327C)* | | gel (PFG 327C)* | |
| K | Dark green clear gel | No evidence of | Dark green clear gel | No evidence of |
| | (PFG 327C)* | copper usnate | (PFG 327C)* | copper usnate |
| | | crystals | | crystals |
| LP | Small agglomerates | n/a | Small agglomerates | n/a |
| | observed in a pale | | observed in a pale | |
| | yellow clear gel | | yellow clear gel | |
| | (PFG 1205C)* | | (PFG 1205C)* | |
| KP | Pale yellow clear gel | n/a | Pale yellow clear gel | n/a |
| | (PFG 1205C)* | | (PFG 1205C)* | |

*Colours determined from the Pantone Formula Guide (PFG)

1. A topical skin treatment formulation containing usnic acid or an usnate salt dissolved in a solvent system, wherein the solvent system comprises:
(i) dimethyl isosorbide (DMI);
(ii) a C1 to C9 alkyl salicylate; and
(iii) a glyceryl fatty acid ester.

2. The formulation of claim 1, wherein the usnate salt is copper usnate.

3. The formulation of claim 1, wherein the solvent system additionally comprises (iv) an alcohol.

4. The formulation of claim 1, wherein the solvent system additionally comprises (v) a polyoxyalkylene-based solvent selected from polyoxyalkylene glyceryl esters, polyalkylene glycols, and mixtures thereof.

5. The formulation of claim 1, wherein the solvent system additionally comprises a C1 to C4 alkyl glucose ester.

6. The formulation of claim 1, wherein the solvent system comprises:
(1) DMI, at a concentration from 25 to 35% w/w;
(2) homosalate, at a concentration from 5 to 12% w/w;
(3) a glyceryl fatty acid ester selected from PCA glyceryl oleate, glyceryl diisostearate and mixtures thereof, at a concentration from 2 to 8% w/w;
(4) an alcohol selected from ethanol, THFA and mixtures thereof, at a concentration from 15 to 25% w/w;
(5) a component selected from polyoxyethylene glyceryl esters, PEGs, and mixtures thereof, at a concentration from 22 to 32% w/w; and
(6) a methyl glucose ester, at a concentration from 2 to 8% w/w.

7. The formulation of claim 1, wherein the concentration of the usnic acid or usnate salt in the formulation is from 0.1 to 2% w/w.

8. The formulation of claim 1, which comprises salicylic acid or a derivative thereof.

9. The formulation of claim 1, which is in the form of a solution, lotion, cream or gel.

10. A product which incorporates the formulation of claim 1.

11-13. (canceled)

14. A method of treatment of a human or animal patient suffering from or at risk of suffering from a skin or skin structure condition, or of a condition which is caused by, transmitted by and/or exacerbated by microbial activity, the method involving administering to the patient a therapeutically effective amount of the formulation of claim 1.

15. (canceled)

16. The method of claim 14, wherein the skin or skin structure condition is acne.
